(19) United States
(12) Patent Application Publication
(10) Pub. No.: US 2026/0137423 A1
(43) Pub. Date: May 21, 2026

(54) DEVICE FOR EXTRACTING ELEMENTS FROM A CAVITY AND METHOD OF OPERATION

(71) Applicant: Maternal Newborn Health Innovations, PBC, Parsippany, NJ (US)

(72) Inventors: Mario Merialdi, Geneva (CH); Benjamin Bringuier, Singapore (SG); Anna Franca Cavaliere, Rome (IT); Michel Boulvain, Thônex (CH); Sze Jiunn Ang, Singapore (SG)

(21) Appl. No.: 19/289,383

(22) Filed: Aug. 4, 2025

Related U.S. Application Data
(60) Provisional application No. 63/679,682, filed on Aug. 6, 2024.

(51) Int. Cl. A61B 17/44; A61B 17/00

(52) U.S. Cl. CPC A61B 17/442; A61B 2017/00137; A61B 2017/00424; A61B 2017/00544; A61B 2017/00561

(57) ABSTRACT

A birth assist device having an open-ended sleeve with an inflatable chamber that is guidable by a clinician. The open-ended sleeve has a handle affixed to a proximal end thereof and a clinician gripping structure at a distal end thereof. The clinician gripping structure may be a hand extender, one or more finger tabs or one or more finger pockets, or some combination thereof. The hand extender receives some portion of the hand of the clinician while each finger tab or pocket receives an individual finger of a clinician. The inflatable chamber is in fluid communication with an inflation device, which may be a bulb pump. The distal end of the sleeve is sized to securely fit on the upper portion of a baby's head. Also described is a method for assisting a Caesarean Section birth using the birth assist device.

### CROSS REFERENCE TO RELATED APPLICATIONS

This application claims the benefit of and priority to U.S. Provisional Application No. 63/679,682 which was filed on Aug. 6, 2024, and which is incorporated by reference herein in its entirety.

### TECHNICAL FIELD

A device to extract elements contained in a cavity and operation thereof is described. The proposed device is preferably used to assist at childbirth and/or to extract elements seated in cavities of the human body, facilitating the task of medical professionals.

### BACKGROUND

There have been several innovations in the field of child birth which aid in the child bearing process even in situations when healthcare professionals/facilities are not available. Some of these innovations are related to minimizing potentially fatal maternal complications (for example, hemorrhages, infection) and newborn complications (for example, birth asphyxia, trauma) during the second stage of labor.

One example of such an innovation is the Odon Assist™ (which is a trademark of MATERNAL NEWBORN HEALTH INNOVATIONS, PBC) which is shown in FIG. 1. The device may be broadly described as an instrument to deliver a fetus when complications occur during the second stage of labor. The device includes a sleeve with an inflatable section made of film-like polyethylene material and operates in a different manner to a forceps and vacuum extractor for assisted deliveries. The device illustrated in FIG. 1 is described in U.S. Pat. Nos. 11,097,992, 11,690,650 and 11,986,217 to Koh et al., the disclosure of which is incorporated by reference herein. The device illustrated in FIG. 1 includes an inflator that is attached to the birth assist device via a conduit to supply a positive pressure that inflates an inflatable section of the birth assistance device. The apparatus includes a central chamber for containing a gas, the central chamber including a first compressible portion and a second incompressible portion. The pressure limitation apparatus may also be configured to operate with a birth assistance device when attached via a conduit to supply a negative pressure to withdraw air from within a suction cup of the birth assistance device. U.S. Pat. No. 9,247,958 also describes the Odon Assist and the disclosure of which is incorporated by reference herein.

The presently disclosed device and method aims to enhance the Odon Assist.

### BRIEF SUMMARY

The device described herein is configured for use to assist childbirth by Caesarean Section. Caesarean section (C-S) is a commonly performed procedure in obstetrics practice. One of the major advantages of C-S delivery is that the fetal passage through the birth canal is eliminated and a controlled fetal delivery under direct visualization may be achieved. However, there are some C-S deliveries where fetal extraction is difficult to perform such as cases of “floating head” where the fetal head is above the pelvic inlet. For such deliveries, obstetricians heretofore may use prior art devices such as the forceps or the vacuum to properly as securely deliver the fetus in this position. The device described herein provides improvements over the prior art devices.

The device described herein is initially comprised of a plastic bag or sleeve which has, at a distal end, one or more finger tabs configured to receive one or more fingers of a clinician. The distal end of the plastic sleeve is placed over baby's head and the one or more finger tabs permit the clinician to guide the bag or sleeve over the baby's head (i.e., the cephalic pole) to assist in removing the baby from the mother's womb. The one or more finger tabs allow the clinician to guide the sleeve onto the fetal neck or chin or jaws, depending on the fetal position. The distal end of the sleeve also may have an extender that is configured to assist the clinician in extending the sleeve into position over the baby's head. The extender may be affixed to the sleeve, but with a gap between the sleeve and the extender that allows the clinician to slide their hand through the gap for placement between the extender and the sleeve. The back of the clinician's hand faces outward and rests against the extender, which is configured to provide leverage to the clinician's hand to permit the clinician to firmly guide the sleeve into the desired position. The extender may have different configurations, but provides support for one or more fingers of the clinician's hand. The one or more finger supports are referred to as flat bone strips herein. The one or more finger supports extend distally along the sleeve exterior from a base strip. The base strip extends laterally along the sleeve perimeter. The base strip is affixed to the sleeve at points sufficiently spaced apart to allow the insertion of the clinician's hand between the affixation points to allow the back of the clinicians fingers to rest against the one or more flat bone strips.

The sleeve also has an air chamber at its distal end that, when inflated, assists it gripping the baby's head. The device also has an inflation device associated therewith to inflate the air chamber. The proximal end of the plastic sleeve is closed and has a handle attached or coupled thereto. Since the proximal end of the sleeve is open, the handle may be in two parts, each attached to the proximal end of the sleeve opposite each other. The two parts of the handle may be brought together and held by the clinician while manipulating the device.

In one aspect, the sleeve extends distally beyond the air chamber. The extender and the finger tap are mounted on the interior of the sleeve but are accessible to the clinician when the sleeve is folded over at its distal end. The distal folded over portion of the sleeve is advanced over the baby's head during placement. Further in this regard, the extender may be located within the fold when the device is deployed. The clinician may place their hand within the fold and advance their hand into position in the extender. At this time, the clinician then begins to extend the distal portion of the sleeve into position over the baby's head to assist the clinician with delivering the baby from the womb.

The bag may have a feature to prevent the air chamber from being over inflated. The bag may have a deflation device, such a button, for emergency deflation of the air chamber.

Said sleeve may be made of extremely flexible material, film-type of some microns wide, with a highest traction resistance, said material being hypoallergenic. In one aspect, the sleeve may be covered with fine lubricating gel. The sleeve may be asymmetrical at its distal end, with the sleeve portion to which the extender is attached being longer than the sleeve portion to which the one or more finger tabs are attached.

Said open sleeve with its integrated air chamber around its distal end shall be inserted by the medical professional between the head of the fetus and cavity (e.g., the uterus) from which the fetus is to be retrieved. To accomplish this, the clinician inserts one of more of their fingers into the one or more finger tabs to first align the sleeve with a portion of the baby's head that may be exposed through the incision through which the baby will be delivered. The clinician will then place their other hand into the extender, the back of one or more of their fingers resting against one or more of the flat bone strips, to guide the distal end of the sleeve over baby's head. The extender is configured so that the clinician may remove their hand and then, pushing against the base strip, advancing the extender to position the sleeve over the baby's head for delivery. In some aspects, the bag will be adjusted so that the distal end is at an angle with respect to the proximal end (i.e. the handle end) of the sleeve. The clinician then grasps the handle with their free hand. The air chamber is then inflated, further gripping baby's head. Upon inflation, the clinician continues to grasp the handle. The clinician then pulls the baby from the cavity.

In one aspect, the air chamber shall comprise a regulating and compensating valve for the inlet of air or another fluid of very low pressure, which valve shall be calibrated to act as an air outlet valve pursuant to the adjustment selected by the gynecological professional.

It is important to maintain a controlled inner pressure in the air chamber since the value of said pressure may increase when extracting the flexible element from the interior of the cavity up to values that may complicate the extraction due to the increase of volume that causes this greater generated pressure. Said regulating valve shall be a relief valve for this generated pressure, avoiding that said pressure raises beyond a predetermined value.

### BRIEF DESCRIPTION OF DRAWINGS

The device described herein is illustrated in the following figures:

FIG. 1 illustrates a perspective view of a prior art device for the extraction of elements of the device described herein;

FIG. 2 illustrates a first example of a sleeve inflation device from the prior art;

FIG. 3 illustrates a second example of a sleeve inflation device from the prior art;

FIG. 4 illustrates a third example of a sleeve inflation device from the prior art;

FIG. 5(a), FIG. 5(b) and FIG. 6 are schematics illustrating operation of the prior art inflation device; and

FIG. 7 to FIG. 9 are additional illustrations of the principals of operation for the prior art inflation device.

FIG. 10 to FIG. 13 are additional illustrations of the principals of operation for the prior art inflation device.

FIG. 14 illustrates a birth assist device for a Caesarean Section birth;

FIG. 15 is a bottom up cut away view along line A-A of FIG. 14;

FIG. 16 is a detail view of a portion of the device illustrated in FIG. 15;

FIG. 17 illustrates an aspect of an extender for the birth assist device described herein;

FIG. 18 illustrates the extender according to a different aspect;

FIG. 19 illustrates the extender according to a different aspect;

FIG. 20 illustrates the extender according to a different aspect;

FIG. 21 illustrates the extender according to a different aspect;

FIG. 22 illustrates the extender according to a different aspect;

FIG. 23 illustrates an alternative aspect of a birth assist device for a Caesarean Section birth;

FIG. 24 illustrates an alternative aspect of a birth assist device for a Caesarean Section birth;

FIG. 25 illustrates an alternative aspect of a birth assist device for a Caesarean Section birth;

FIG. 26A to FIG. 26D illustrate one method for using the birth assist device described herein;

FIG. 27 illustrates an alternative embodiment of the birth assist device described herein;

FIG. 28(a) to FIG. 28(d) illustrate the use of the birth assist device in FIG. 26 for a Caesarean Section birth; and

FIG. 29 illustrates how the device of FIG. 25 receives the fingers of the clinician in the sleeve.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

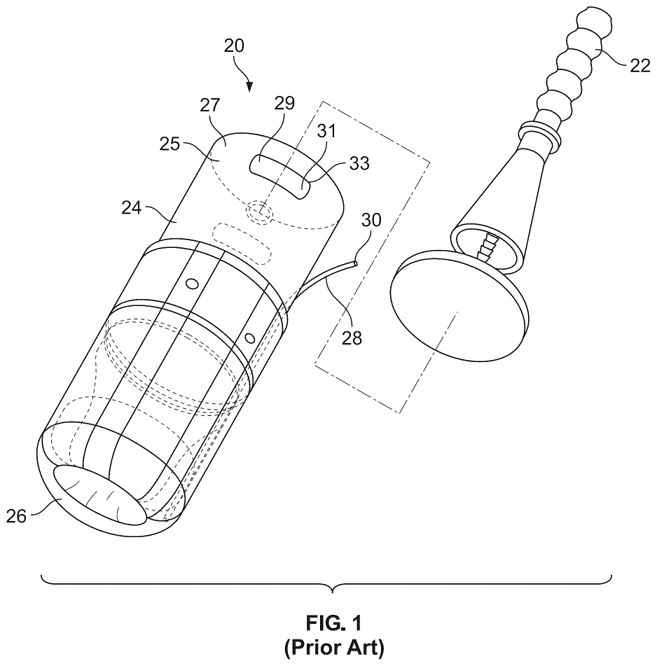

In the FIG. 1 prior art device, there is shown a perspective view of various components of an apparatus 20 (the Odon Assist) for extraction of at least one element from a cavity. Only main components of the apparatus 20 are described. The apparatus 20 is for assisting the extraction of a baby from a vagina of a female during the child-birth process, and is used in certain situations when the female encounters difficulty during the child-birth process. The apparatus 20 includes a central inserter 22 which is removed after the apparatus 20 is appropriately positioned in the vagina of a female. The apparatus 20 also includes a sleeve 24. The sleeve 24 includes an inflatable section 26 configured to surround the at least one element during inflation, made of film-like polyethylene material. Ingress of air into the inflatable section 26 is via an inflation tube 28, and typically, a pump (not shown) is attached to the inflation tube 28 at a first end 30. The sleeve 24 also includes a handle 29 for a user to hold onto when removing the sleeve 24 from the vagina. The sleeve 24 includes an opening 31 at a first portion 25 of the sleeve 24, the opening 31 being configured to allow passage of the user's fingers when the user is holding onto the handle 29. The handle 29 defines a holding edge 33 of the opening 31.

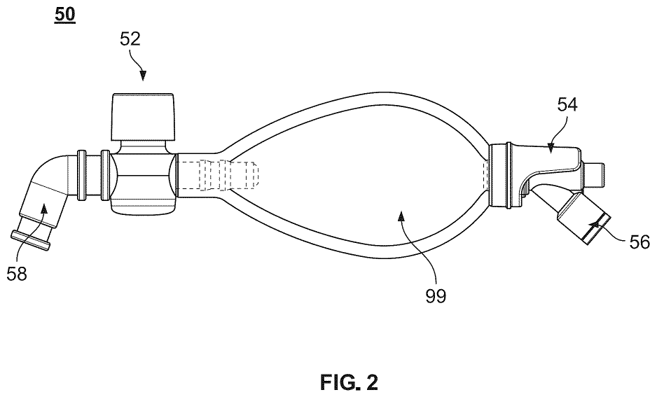

Additional aspects of the handle 29 are described in U.S. Pat. Nos. 11,097,992, 11,690,650 and 11,986,217 to Koh et al., which are incorporated by reference herein. The handle may include a pump that will introduce air into the sleeve 24 to inflate the inflatable section 26 via inflation tube 28 at its first end 30. One example of such a pump assembly 50 is illustrated in FIG. 2, which illustrates central chamber 99 in a bulb pump configuration. The bulb 99 (shown in phantom) is part of a pump assembly 50 which is configured to connect to the inflation tube 28 at its outlet end via connector 58. The pump may have a deflation assembly 52 (i.e., a switch) on its outlet end and a securement ring 54 and pressure limiter 56 on its inlet end. The user may inflate the air chamber via hand compression of the bulb 99. The air chamber 26 may form a “gripper” on the fetal head with pressure regulated by the pressure limiter 56. The baby may be extracted by pulling the handle 29 on the sleeve 24.

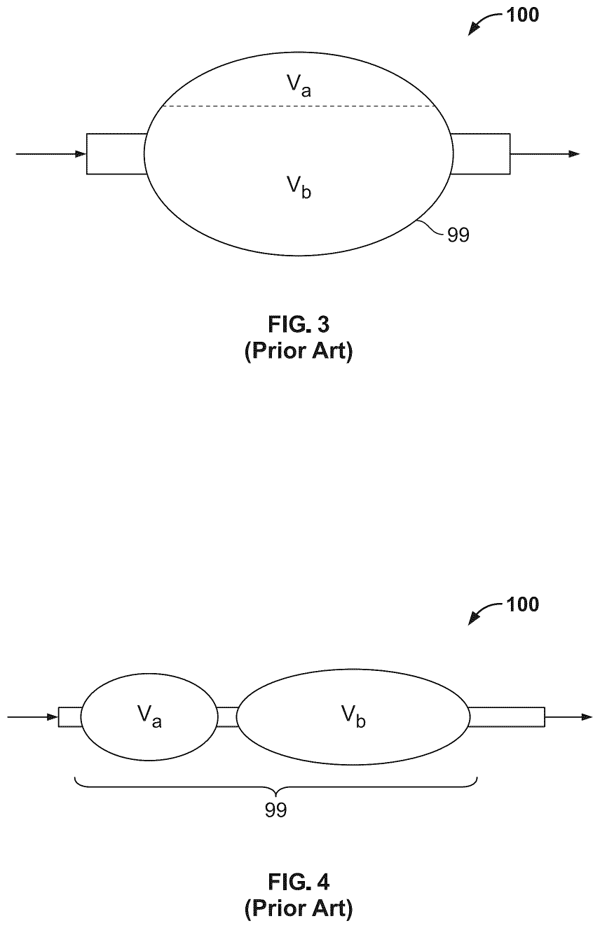

In one aspect of the prior art, the bulb 99 may contain incompressible and compressible volumes. This prior art configuration may be used with the device described herein, but pumps that do not require incompressible and compressible volumes are suitable for use with the device described herein. Referring to FIG. 3 to FIG. 11, there is shown a pressure limited pump 100 of the prior art. FIG. 3 shows use of an external guide (restrictor) mounted to the central chamber 99 to define the compressible portion, Va, and incompressible portion, Vb. FIG. 4 shows use of a separate compressible portion (a first sub-chamber), Va, and a separate external incompressible portion (a second sub-chamber), Vb to make up the central chamber 99.

FIG. 3-FIG. 4 depict the central chamber 99 in a bulb pump configuration. FIG. 3 shows use of an internal partition (such as, for example, partitions, stoppers, membranes, and the like) within the central chamber 99 to define the compressible portion, Vₐ, and incompressible portion, V_{b}. FIG. 3 shows use of an external guide (restrictor) mounted to the central chamber 99 to define the compressible portion, Va, and incompressible portion, V_{b}. FIG. 4 shows use of a separate compressible portion (a first sub-chamber), Vₐ, and a separate external incompressible portion (a second sub-chamber), V_{b} to make up the central chamber 99.

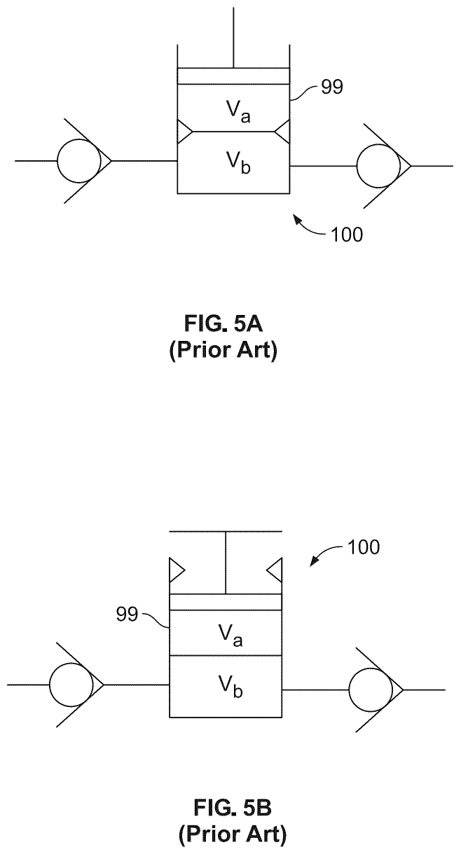

FIG. 5(a), FIG. 5(b) and FIG. 6 depict the central chamber 99 in an axial pump configuration. FIG. 5(a) shows use of objects within the central chamber 99 to define the compressible portion, Vₐ, and incompressible portion, V_{b}. FIG. 5(b) shows use of an external guide (restrictor) mounted to the central chamber 99 to define the compressible portion, Vₐ, and incompressible portion, V_{b}. FIG. 6 shows use of a separate compressible portion, Vₐ, and a separate external incompressible portion, V_{b} to make up the central chamber 99. It should be appreciated that other variations for the central chamber 99 are also possible. Moreover, it should be appreciated that in all the embodiments shown in FIG. 2 to FIG. 6, Vₐ and V_{b} remain in fluid communication.

It should be appreciated that the pressure limited pump 100 may be used to apply a positive pressure to supply air to an inflatable section 26 like that of the apparatus 20, and that it may also be used to apply a negative pressure to withdraw air from within an enclosed region (like a suction cup of a ventouse). It is critical to limit the negative pressure so as to minimize damage to a baby's head during delivery of the baby. Thus, the pressure limited pump 100 enables limitation of the negative pressure without the user's intervention, that is, without needing to monitor the pressure level within the enclosed region. Moreover, use of the pressure limited pump 100 results in redundancy of a pressure gauge and a pressure releaser which is typically used with the ventouse. Thus, a number of components which are required when using the ventouse is also minimized, correspondingly minimizing cost and device complexity.

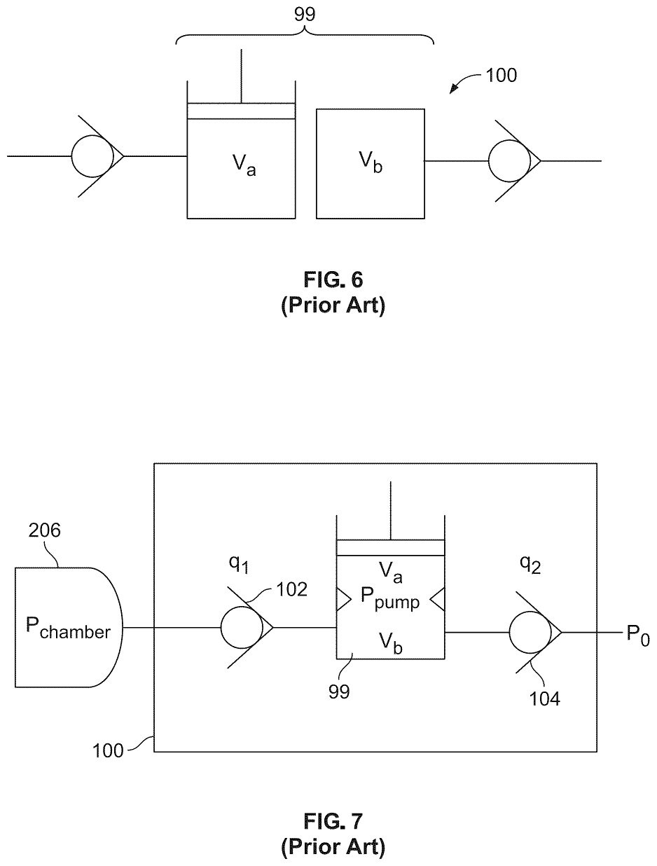

In the instance when the pressure limited pump 100 is used to apply a negative pressure, referring to FIG. 7 to FIG. 9, FIG. 7 shows an initial state of the pressure limited pump 100 which includes a central chamber 99, a first check valve 102 and a second check valve 104. The central chamber 99 is a compressible portion (Vₐ) and incompressible portion (V_{b}) which serves as pressure limit for the pressure limited pump 100. The first check valve 102 allows air to pass through when the central chamber 99 pressure (Pₚᵤₘₚ) is higher than atmospheric pressure (P₀). It also prevents back flow of air when air is being pressurized within the central chamber 99. The second check valve 104 allows air to pass through when Pₚᵤₘₚ is higher than air chamber 206 pressure (P_{chamber}). The air chamber 206 represents the suction cup of the ventouse. At the initial state, no air is flowing through the first check valve 102 and the second check valve 104. Thus, q₁=q₂=0, and P_{chamber}=Pₚᵤₘₚ=P₀.

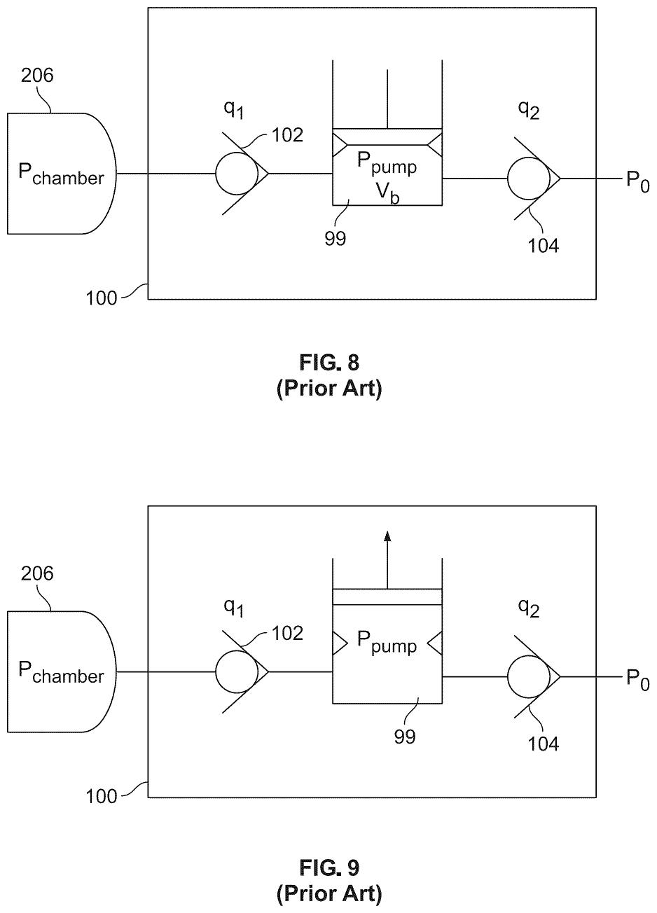

FIG. 8 shows compression of the central chamber 99, leading to air passing through the second check valve 104 into the atmosphere. At this juncture, Pₚᵤₘₚ>P₀.

After FIGS. 7 and 8, FIG. 9 shows the central chamber 99 drawing air from the air chamber 206, thus increasing internal volume of the central chamber 99 and leading to air passing through the first check valve 102 into the central chamber 99.

The following paragraphs provide theoretical aspects in relation to the workings of the pressure limited pump 100 that are described in the prior art. By controlling a ratio of compressible volume (Vₐ) and incompressible volume (V_{b}) in the central chamber 99, a maximum negative pressure that may be delivered by the central chamber 99 may be designed (defined) using Boyle's Law as follows:

In FIG. 7, Pₚᵤₘₚ V_{b}=P₀(Vₐ+V_{b}).

In FIG. 9, P_{pump initial}V_{b}=P_{pump final}(Vₐ+V_{b}), so P_{pump final}=P_{pump initial}×[V_{b}/Vₐ+V_{b}].

Therefore, P_{pump final}<P_{pump initial};
assuming P_{chamber}=P₀ at FIG. 8, P_{pump final}<P_{chamber};
repeating FIG. 8 and FIG. 9 until P_{pump final}=P_{chamber}, this indicates that by managing Vₐ and V_{b}, a pressure limit for P_{chamber} may be obtained.

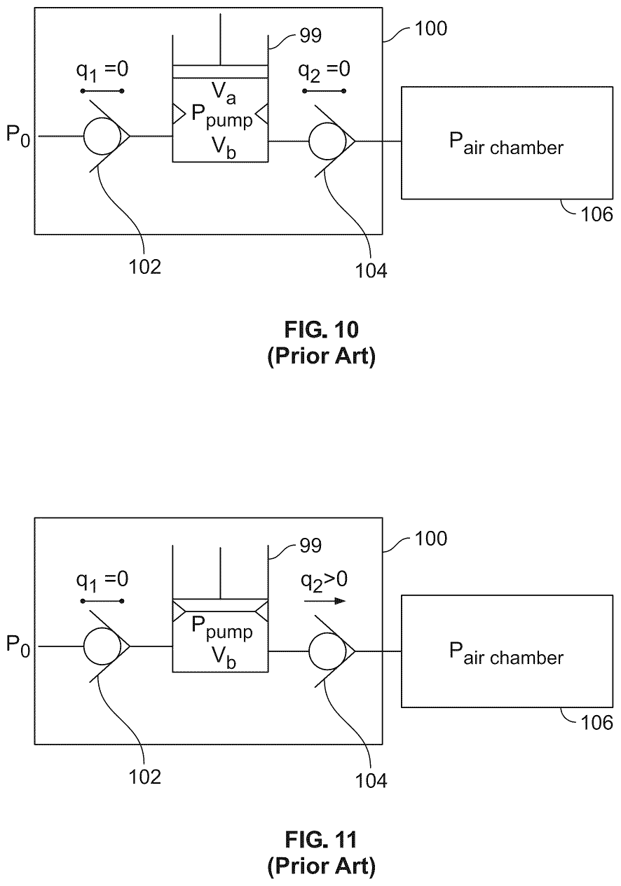

FIG. 10 to FIG. 13 illustrate schematic diagrams of a pressure limited pump 100 during use to supply positive pressure. It should be appreciated that the pressure limited pump 100 may be used with other birth assistance devices such as, for example, a ventouse. The pressure limited pump 100 is typically attached to the birth assistance device via a conduit. The pressure limited pump 100 may also be viewed as a pressure limitation apparatus. In addition, the pressure limited pump 100 is configured for operation involving gases.

FIG. 10 shows an initial state of the pressure limited pump 100 which includes a central chamber 99, a first check valve 102 and a second check valve 104. The central chamber 99 is for drawing air from atmosphere and expelling the air, and includes a compressible portion (Vₐ) and incompressible portion (V_{b}) which serves as pressure limit for the pressure limited pump 100. The first check valve 102 allows air to pass through when the central chamber 99 pressure (Pₚᵤₘₚ) is lower than atmospheric pressure (P₀). It also prevents back flow of air when air is being pressurized within the central chamber 99. The second check valve 104 allows air to pass through when Pₚᵤₘₚis higher than air chamber 106 pressure (P_{air chamber}). The air chamber 106 represents the inflatable section 26 of the sleeve 24. The second check valve 104 also prevents back flow of air from the air chamber 106 when air is being drawn from atmosphere into the central chamber 99. At the initial state, no air flow through the first check valve 102 and the second check valve 104. Thus, q₁=q₂=0, and P_{air chamber}=Pₚᵤₘₚ=P₀.

FIG. 11 shows compression of the central chamber 99, leading to air passing through the second check valve 104 into the air chamber 106. At this juncture, Pₚᵤₘₚ>P_{air chamber} and q₂>0. Subsequently, P_{air chamber} increases due to displacement of air into the air chamber 106.

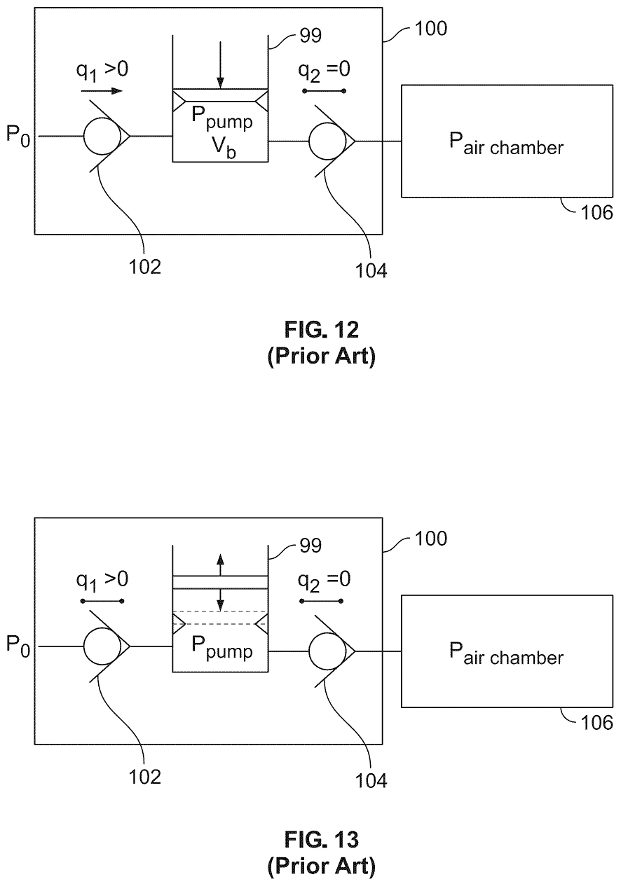

After FIG. 11, FIG. 12 shows the central chamber 99 drawing air from the atmosphere, thus increasing internal volume of the central chamber 99 and leading to air passing through the first check valve 102 into the central chamber 99. Thus, Pₚᵤₘₚ<P₀, q₁>0, Pₚᵤₘₚ<P_{air chamber} and q₂=0.

When steps depicted in FIG. 11 and FIG. 12 are repeated (that is, repeated pump-release actions on the central chamber 99), a juncture will be reached whereby pressure equilibrium is reached, that is Pₚᵤₘₚ=P_{air chamber}, and q₁=q₂=0 since there is no air flow between any of the central chamber 99, the air chamber 106 and the atmosphere.

The following paragraphs provide theoretical aspects in relation to the workings of the pressure limited pump 100.

When pressure within the central chamber 99 (Pₚᵤₘₚ) is equal to the pressure of the air chamber (P_{air chamber}), no air will be transferred at the second check valve 104, as shown in FIG. 13.

Thus, by controlling a ratio of compressible volume (Vₐ) and incompressible volume (V_{b}) in the central chamber 99, a maximum pressure that may be delivered by the central chamber 99 may be designed (as defined using Boyle's Law) as follows:

$$Boyle ’ s Law : {P}_{1} {V}_{1} = {P}_{2} {V}_{2} \qquad ( a )$$

$$Initial Pressure : {P}_{1} = {P}_{0} \qquad ( b )$$

$$Initial Volume : {V}_{1} = {V}_{a} + {V}_{b} \qquad ( c )$$

$$Final Volume : {V}_{2} = {V}_{b} ( that is , the incompressible volume ) \qquad ( d )$$

Therefore,

$$\begin{matrix}( e is derived from equation a ) & \\ {P}_{2} = {P}_{1} \times {V}_{1} / {V}_{2} & \end{matrix}$$

Substitute (b), (c), (d) into (e);

$${P}_{2} = {P}_{0} \times ( {V}_{a} + {V}_{b} ) / {V}_{b}$$

$${P}_{2} = Final Pressure = Maxmimum Pressure Delivered = Pressure Limit$$

$$When , {P}_{air chamber} = {P}_{pump} = {P}_{2}$$

$${q}_{2} = 0$$

Therefore, by defining the ratio between the compressible volume, Vₐ, and incompressible volume, V_{b}, the maximum pressure delivered to the air chamber 106 may be limited. The pressure limited pump 100 ensures that the pressure in the inflatable section 26 is not excessive during use of the apparatus 20.

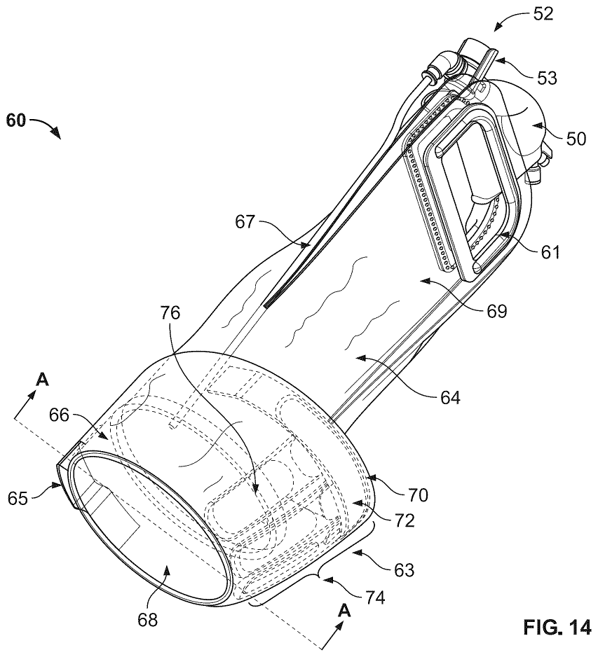

FIG. 14 illustrates one aspect of the device 60 described herein. The device 60 may have a sleeve bag 64 made of a flexible material that has a proximal open end 69 and a distal open end 68. Within the distal end 68 of the sleeve bag 64 is an air chamber 66 connected to tubing 67, which may be linked to the handle assembly 61 on the proximal end 69 of the sleeve bag. The handle assembly 61 consists of a bulb pump assembly 50 that manages the air flow and pressure of the sleeve bag 64 and, more specifically the inflatable chamber 66. The bulb pump assembly 50 may be positioned on the handle 61 that allows the clinician, with a single grip, to pull the handle, compress the bulb pump 50 and to deflate the air chamber 66 by pressing the push button on the deflation assembly 52. Accidental deflation of the air chamber 66 may be prevented by a guard 53 alongside the deflation assembly 52 on the handle 61.

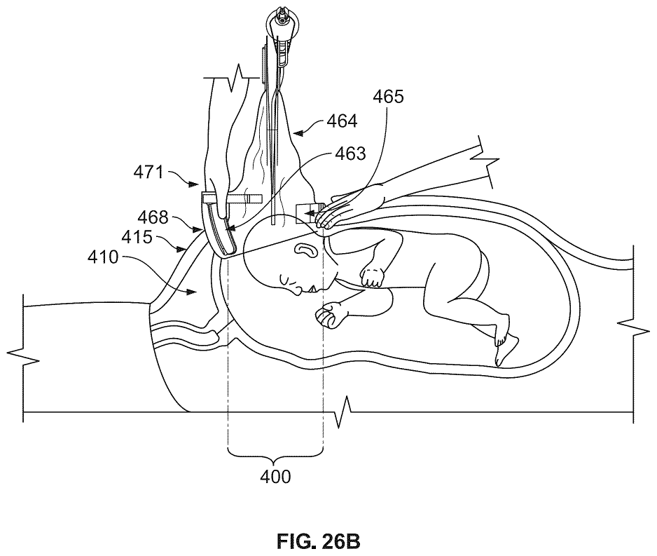

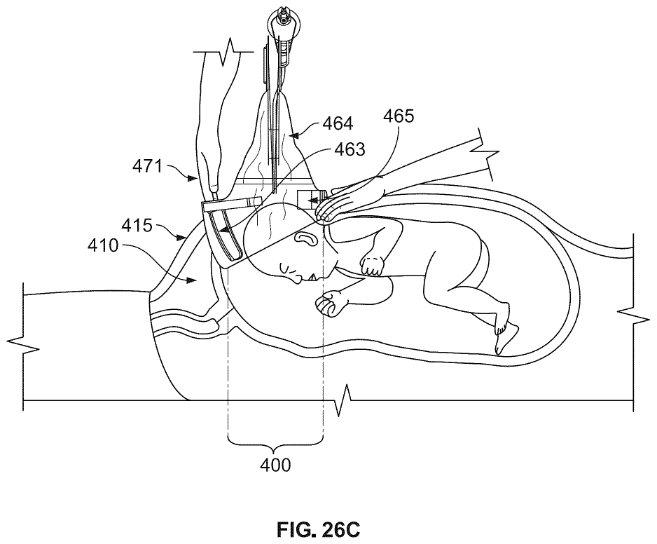

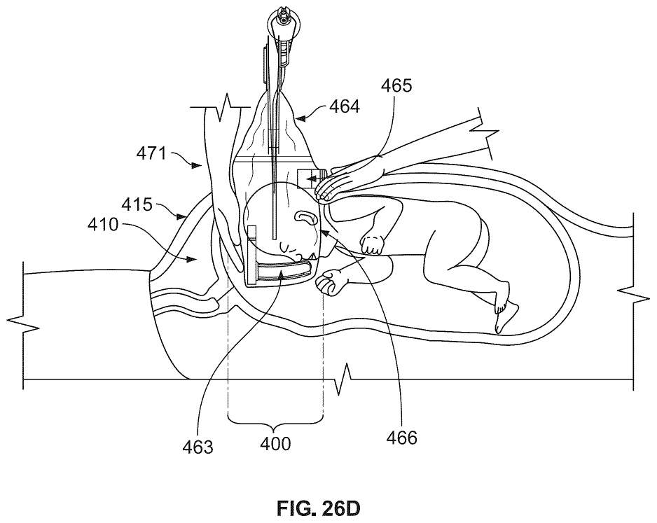

On the distal end 68 of the sleeve 64 may be affixed an extender 63. As illustrated, the extender 63 is at least partially disposed over the air chamber 66. The extender 63 mimics a human hand and provides a mixture of flexibility, rigidity and softness. The extender 63 may be manipulated by a human hand. The clinician inserts a portion of their hand in an opening 70 between the extender 63 and the sleeve 64. The extender 63 may have an elongated base strip 72 with finger portions 74 that extend from the elongated base strip 72. Each finger portion 74 may have segments of flat bone strips 76 for flexibility and rigidity. The extender 63 may be encapsulated within silicone for softness. The finger portions are flat relative to a human finger to facilitate insertion (see the following description of the method (FIG. 26C)) of the extender 63 between the baby's head and the “walls” of the womb. In this regard the extender serves as a wedge to leverage the sleeve 64 (and in particular the air chamber 66 at the distal end of the sleeve 64) over the portion of the baby's head that is facing downward from the incision.

At the distal end 68 of the sleeve 64 are one or more finger tabs 65. At the beginning of the delivery process, the finger tabs 65 will receive one or more fingers on one hand of the clinician. The other hand of the clinician (the “leading hand”) is subsequently received by the extender. The finger tabs 65 allow the clinician to position the distal portion of the sleeve 64 over the portion of the fetal head facing the incision. This distal portion of the sleeve 64 is located on the sleeve approximately opposite the distal portion of the sleeve 64 to which the extender 63 is attached. Following this, the clinician inserts their leading hand in the extender 63. The clinician then uses the extender to work the distal portion of the sleeve 64 that includes the air chamber 66 over the portion of the fetal head that is downward facing in the womb. This fetal head position is merely illustrative. The skilled person is aware that the position of the fetus may vary. The device described herein can be used to deliver a fetus in any position in the womb. The relative placement of the components (i.e., extender 63, finger tabs 65, tubing 67, etc.) is merely illustrative. The extender 63 and finger tabs 65 are placed on the distal end so that they be used by the clinician to place the sleeve on the fetal head and assist in delivering the baby from the womb. The tubing 67 is placed so that it will connect the bulb pump assembly 50 to the air chamber 66 and its location is selected such that it will not interfere with the functions of the extender 63 and finger tabs 65.

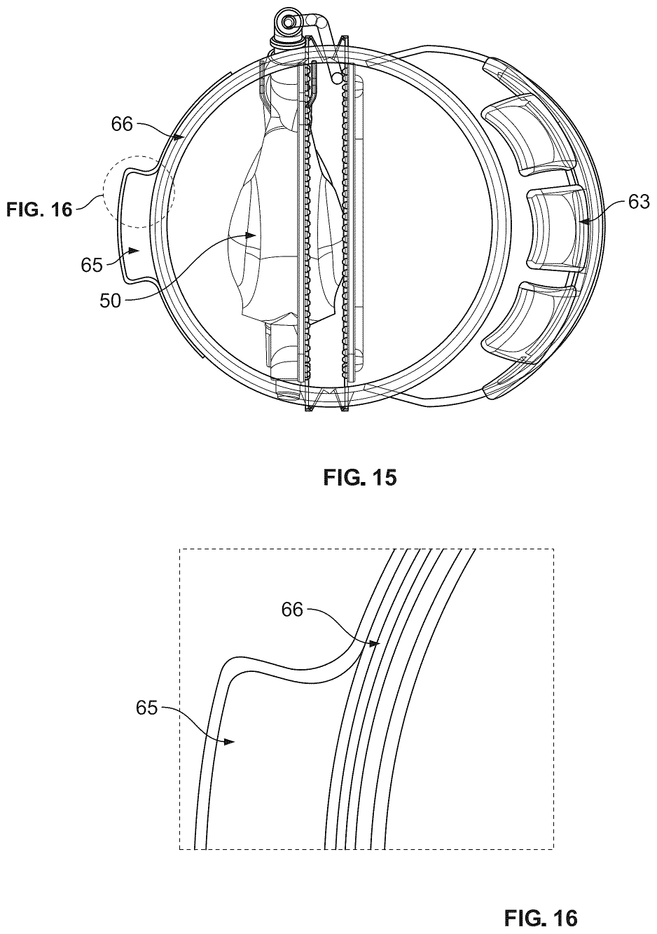

FIG. 15 is a cross-section of the device 60 from the distal end to the proximal end along A-A in FIG. 14. As may be seen, the air chamber 66 is located within the distal end of the sleeve 64. On the exterior of the sleeve 64 at the distal end are the finger tab[s] 65 and the extender 63. The pump 50 is visible at the distal end of the device 60. As noted elsewhere herein, the extender may be attached to the outer surface of the sleeve 64 or disposed on an inner surface of the sleeve 64 formed from a fold in the sleeve at the distal end thereof. In those embodiments where the extender 63 is attached to the inner surface of the sleeve 64 and made accessible to the clinician by folding over the distal end of the sleeve 64. The same is true of the one or more finger tabs, which may be attached to the outer surface of the sleeve 64 or on the inner surface and then made accessible to the clinician by folding over the distal portion of the sleeve. FIG. 16 is a detail view from FIG. 15 showing the finger tab(s) 65 and the air chamber 66.

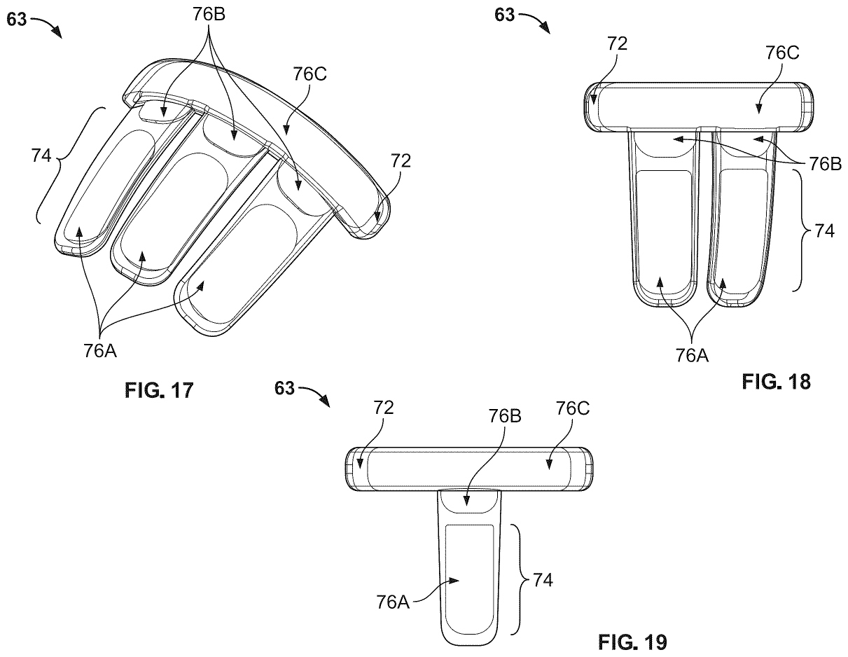

FIG. 17 to FIG. 22 illustrate various embodiments of extender 63. FIG. 17 illustrates an embodiment where extender 63 accommodates three fingers 74. Each finger has two bone strips 76A, 76B. A base strip 72 is configured to form contact with the palm of the hand of the clinician and is also provided with a bone strip 76C.

FIG. 18 illustrates an embodiment where extender 63 accommodates two fingers 74. Each finger has two bone strips 76A, 76B. A base strip 72 is configured to form contact with the palm of the hand of the clinician and is also provided with a bone strip 76C.

FIG. 19 illustrates an embodiment where extender 63 accommodates one finger 74. The finger 74 has two bone strips 76A, 76B. A base strip 72 is configured to form contact with the palm of the hand of the clinician and is also provided with a bone strip 76C.

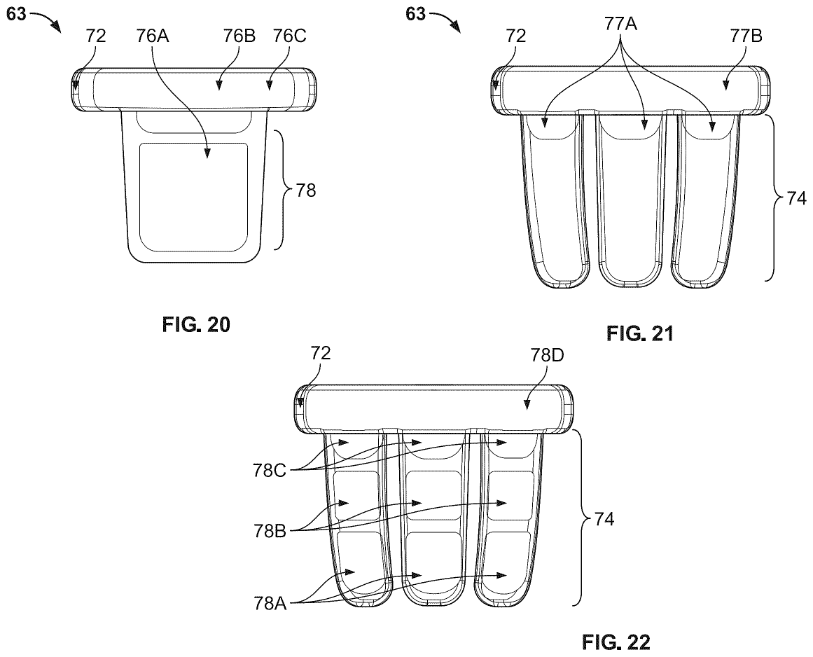

FIG. 20 illustrates an embodiment where extender 63 accommodates more than one finger, but with one finger portion 78. The finger portion 78 has two bone strips 76A, 76B. A base strip 72 is configured to form contact with the palm of the hand of the clinician and is also provided with a bone strip 76C.

FIG. 21 illustrates an embodiment where extender 63 accommodates three fingers 74. Each finger has one bone strip 77A. A base strip 72 is configured to form contact with the palm of the hand of the clinician and is also provided with a bone strip 77B.

FIG. 22 illustrates an embodiment where extender 63 accommodates three fingers 74. Each finger has three bone strips 78A, 78B and 78C. A base strip 72 is configured to form contact with the palm of the hand of the clinician and is also provided with a bone strip 78D.

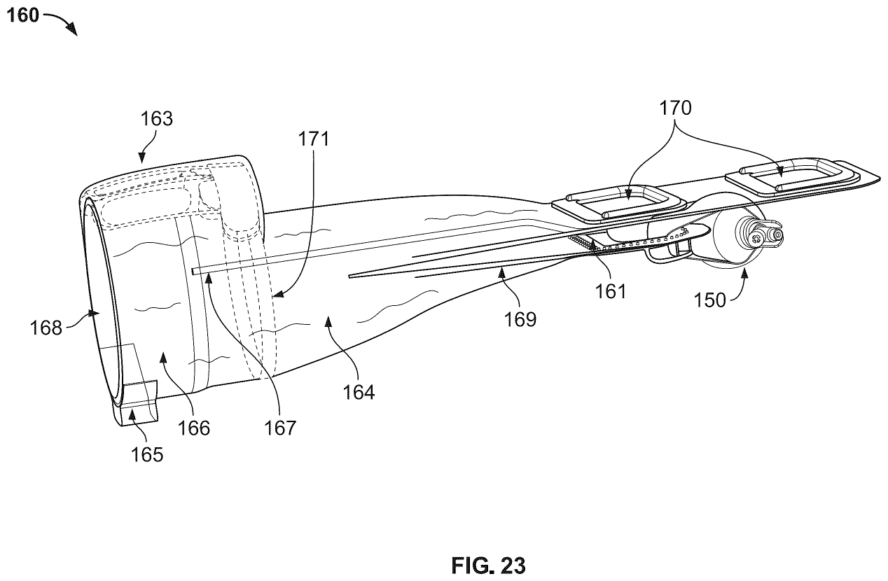

FIG. 23 illustrates an alternative aspect of the device 160 described herein. The device 160 may have a sleeve bag 164 made of a flexible material that has a proximal open end 169 and a distal open end 168. Within the distal end 168 of the sleeve bag 164 is an air chamber 166 connected to tubing 167, which may be linked to the handle assembly 161 on the proximal end 169 of the sleeve bag 164. The handle assembly 161 has multiple handgrips 170 and also consists of a bulb pump assembly 150 that manages the air flow and pressure of the sleeve bag 164 and, more specifically the inflatable chamber 166. The bulb pump assembly 150 may be positioned on the handle 161 that allows the clinician, with a single grip, to pull the handle, compress the bulb pump 150 and to deflate the air chamber 166. Accidental deflation of the air chamber 166 may be prevented by a push-button guard 53 adjacent to push-button switch 52 (FIG. 14) on the handle 161. As in the prior embodiments, the extender 163 and the finger tab(s) 165 are affixed to the distal end 168 of the sleeve 164. In FIG. 23, the sleeve 164 on the extender 163 side is longer than the sleeve on the tab 165 side of the sleeve bag 164 to facilitate positioning the sleeve over the underside portion of the baby's head when the baby is still in the womb. Ring 171 is a circumferential guide so that the clinician may easily see the location of the air chamber 166 in the sleeve 164 and is optional. In some aspects, the position of tubing 167 may be constrained by reinforcement tape (not illustrated).

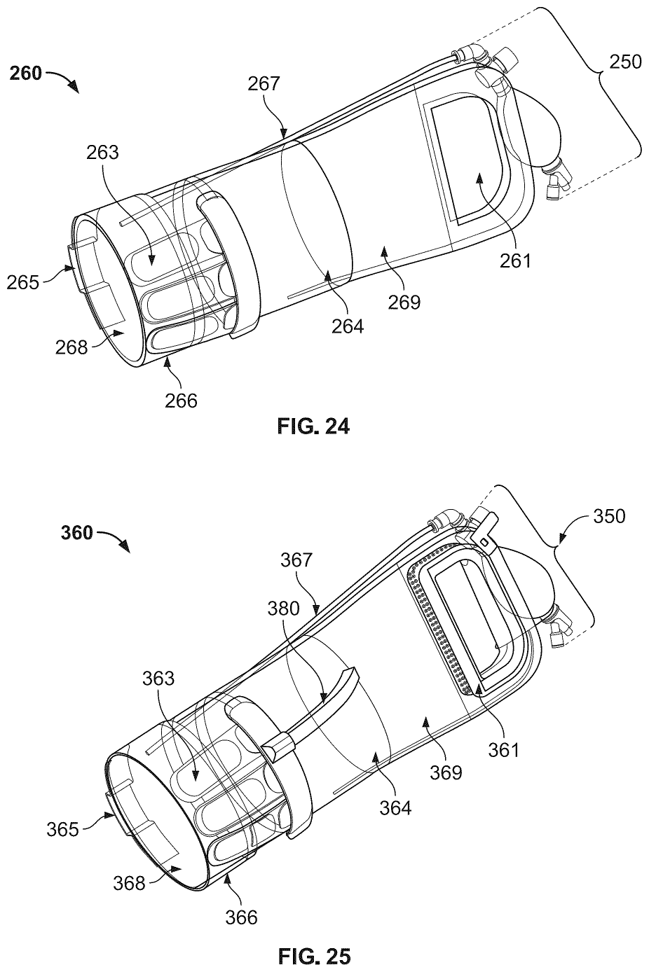

FIG. 24 is an alternate embodiment to what is illustrated in FIG. 23. The device 260 may have a sleeve bag 264 made of a flexible material that has a proximal open end 269 and a distal open end 268. Within the distal end 268 of the sleeve bag 264 is an air chamber 266 connected to tubing 267, which may be linked to an opening 261 in the sleeve that functions as a handle on the proximal end 269 of the sleeve bag 264. A bulb pump assembly 250 that manages the air flow and pressure of the sleeve bag 264 and, more specifically the inflatable chamber 266, but is not attached to the handle opening 261. As in the prior embodiments, the extender 263 and the finger tab(s) 265 are affixed to the distal end 268 of the sleeve 264.

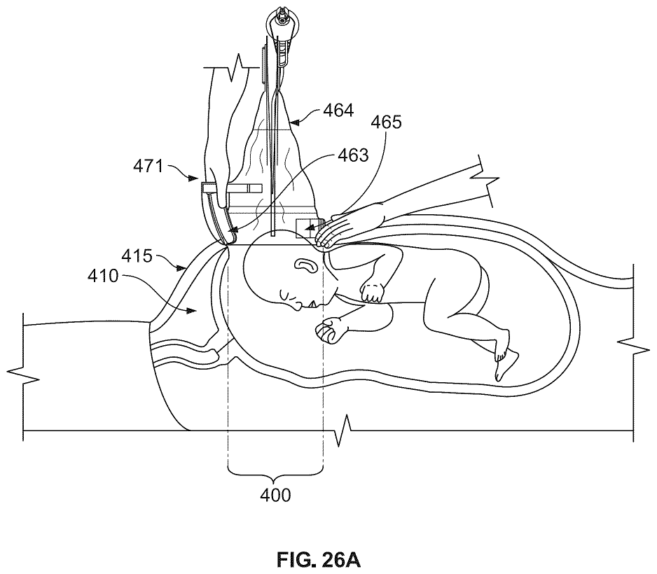

FIG. 25 is an alternate embodiment of the embodiment illustrated in FIG. 23. The device 360 may have a sleeve bag 364 made of a flexible material that has a proximal open end 369 and a distal open end 368. Within the distal end 368 of the sleeve bag 364 is an air chamber 366 connected to tubing 367, which may be linked to the handle assembly 361 on the proximal end 369 of the sleeve bag 364. The handle assembly 361 has a handgrip and also consists of a bulb pump assembly 350 that manages the air flow and pressure of the sleeve bag 364 and, more specifically the inflatable chamber 366. The bulb pump assembly 350 may be positioned on the handle 361 that allows the clinician, with a single grip, to pull the handle, compress the bulb pump 350 and to deflate the air chamber 366. As in the prior embodiments, the extender 363 and the finger tab(s) 365 are affixed to the distal end 368 of the sleeve 364. In this embodiment, a tool 380 is provided to assist the clinician with manipulation of the extender 363.

Operation of the device to assist in the delivery of a baby by Caesarean section using the device illustrated in FIG. 14 is described in FIG. 26A to FIG. 26D. The baby is resting in the womb and the incision 400, through which the baby will be delivered, is illustrated as a gap in the skin 415 layer. As illustrated, the fetal head is facing inward in the womb. This is referred to herein as the OP position, where the fetal neck is exposed through the incision. The device and method described herein are compatible with any fetal head position. Other fetal head positions are, for example, when the chin of the fetal head is facing the incision (OA), or when the cheeks of the fetal head are facing the incision (OT). In FIG. 26A, the clinician manipulates the distal end of the sleeve 464 onto baby's head first using the finger tabs 465. The clinician inserts their fingers into tab(s) 465 openings to guide the sleeve 464 over the neck of the baby. In FIG. 26B, the clinician uses the extender 463 to guide the opposite side of sleeve over the downward facing portion of the fetal head. The clinician inserts one or more of their fingers into a gap 471 (depending on the number of fingers the extender may receive) between the extender 463 and the sleeve to manipulate the extender into the desired position over the downward facing portion of the fetal head. In some aspects, the distal end 468 of the sleeve 464 is asymmetric in that it is longer on the side of the extender 463 than the side of the finger tabs 465. The clinician uses the extender 463 to advance the sleeve 464 into place over the baby's head. Once the sleeve 464 reaches the position where more flexion of the sleeve 464 (˜60° to 90°) is required (or when the clinician needs to bend the hand (˜60° to 90°) to deploy the sleeve 464), the clinician will remove their hand from the pocket and push on the edge of the extender 463 to further deploy the sleeve bag (see FIG. 26C below).

Referring to FIG. 26C, the clinician removes their hand from the extender 463 and pushes on the proximal end of the extender 463 to further advance the sleeve 464 into position on the baby's head. The clinician uses the finger tab(s) 465 to steady the device as the sleeve is advanced into position on the baby's head. In FIG. 26D, once the extender 463 is advanced onto the baby's head as illustrated, the inflatable chamber 466 is inflated to draw the baby from the womb. After delivery, the device is removed from baby and baby is delivered according to normal practice.

The fingers 74 on the extender 463 flex around the contours of the fetal head and deploy the air chamber 466 around the fetal cheeks as illustrated in FIG. 26D. The clinician may then inflate the air chamber 466 via hand compression of the bulb pump 50. The inflated air chamber 466 will form an “air extractor” on the fetal head with pressure regulated by the pressure limiter 56. The baby may then be extracted by pulling the handle 61 on the sleeve bag 64.

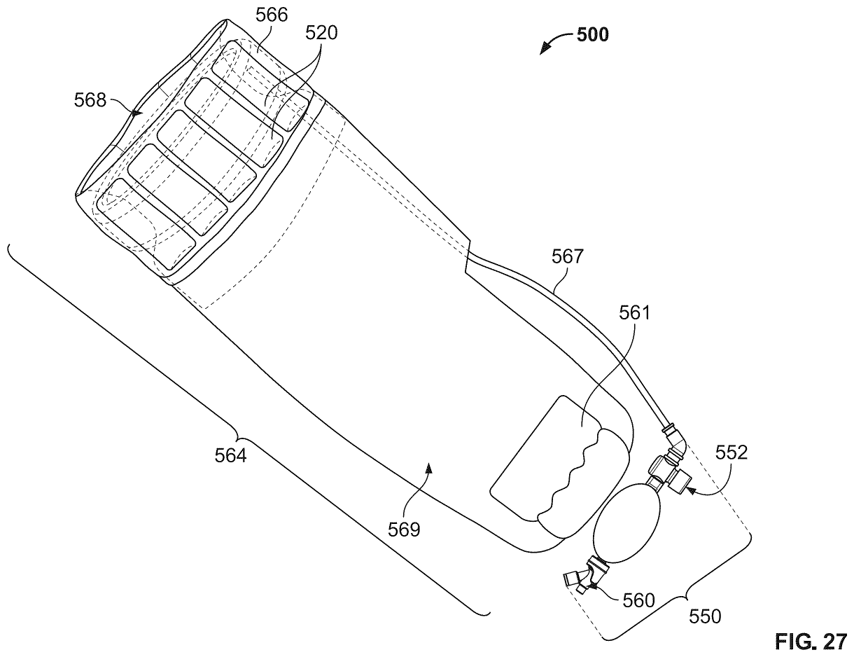

An alternative aspect of the device described herein is illustrated in FIG. 27. The new device is configured to assist with births by Caesarean Section. As illustrated, the plastic sleeve 564 has a distal end of the sleeve 569 that has insertion pockets 520. The clinician may insert their fingers into the insertion pockets 520 to manipulate the distal end 568 of the sleeve 564 onto the baby's head. The distal end 568 of the sleeve 564 also has an air chamber 566 which is configured to inflate when air is received through pump assembly 550. Pump assembly 550 inflates the air chamber 566 via tube 567. Note that, in this embodiment, the inflation device 550 is not integrated with the handle 561. Note that, since the proximal end 569 of the sleeve 564 is open, there is a handle portion 561 on each side of the proximal end 569 of the sleeve 564. Each handle portion 561 may be brought together to form the handle assembly. Brackets and other attachment mechanisms (not shown) may be used to provide structural support to the inflation device. Inflation device 550 is equipped with a decompression valve 560 that prevents over pressure from the inflation device 500. If the air chamber needs to be deflated in an emergency, the clinician may depress a switch 552 on the inflation device 550 to deflate the air chamber 566.

The pockets are configured to receive one finger each. FIG. 27 illustrates five pockets, but the number of pockets is largely a matter of design choice. Two or three pockets to receive two or three fingers, respectively, of each hand are contemplated as suitable.

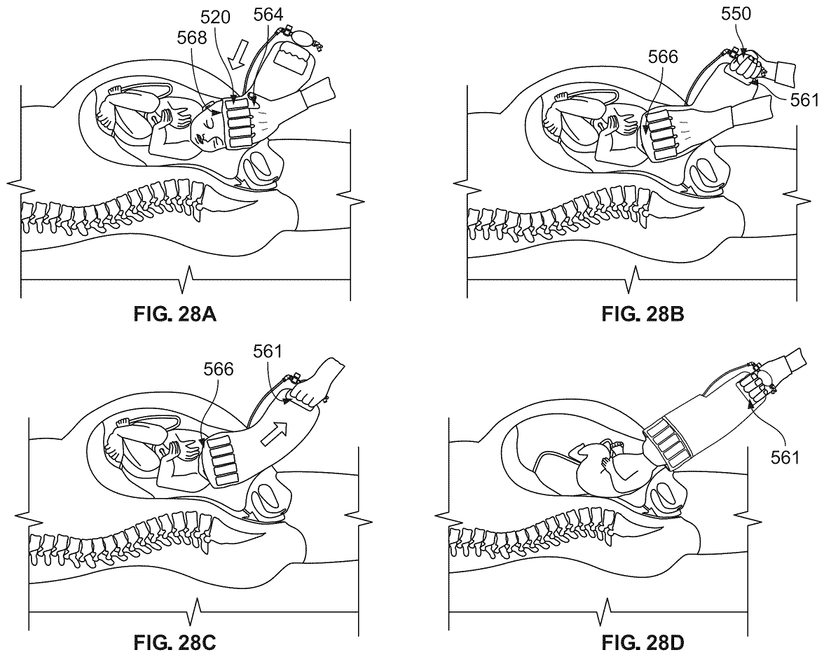

Operation of the device to assist in the delivery of a baby by Caesarean section using the device illustrated in FIG. 27 is described in FIG. 28A to FIG. 28D. In FIG. 28A, the clinician manipulates the distal end 568 of the sleeve 564 onto baby's head. The clinician inserts their fingers into the insertion pockets 520 to grasp baby's head. Baby presents in the womb in a Caesarean Section and therefor the clinician is not reaching through the vagina to grasp baby's head. In FIG. 28B, the clinician inflates the air chamber 566 using the pump 550 and, while maintaining the grasp of baby's head, begins to pull on the handle 561 to draw baby from the cavity.

Referring to FIG. 28C, the air chamber 566 is inflated using inflation device 550 and the clinician releases baby's head to pull on the handle 561. In FIG. 28D, once baby's head is removed from the cavity, the device 500 is removed from baby and baby is delivered according to normal practice.

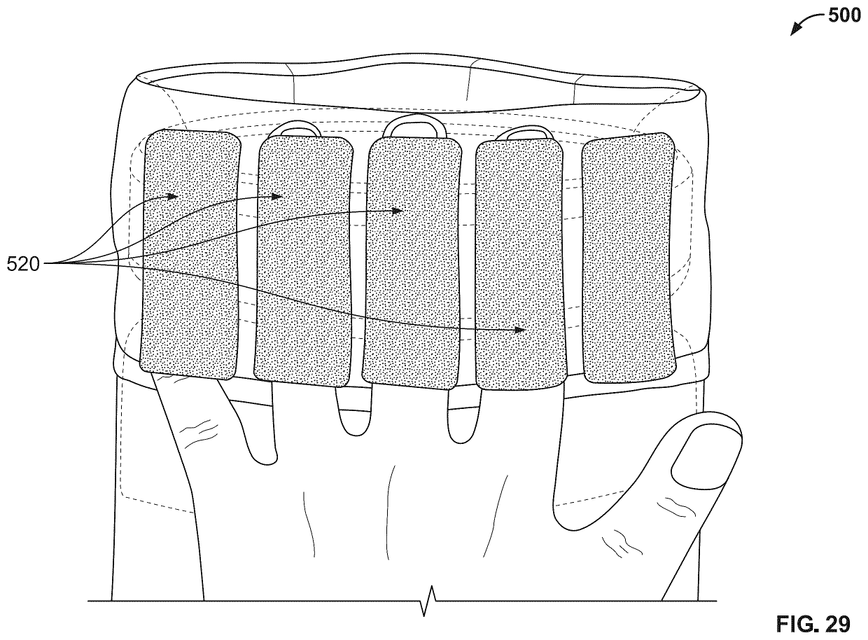

Referring to FIG. 29, the sleeve 564 of the device is illustrated with pockets 520 that receive the fingers of the clinician. This allows the clinician to more easily and consistently secure the sleeve 564 onto the head of the baby. Because the sleeve is inflated, the clinician is not using the pockets to grab the baby's head, but more to guide the placement of the sleeve 564 on the baby's head and to guide the baby out of the mother's womb. The sleeve 564 is fitted with ten pockets 520 to receive the clinician's right hand fingers, left hand fingers, or both. The pockets are provided on the external perimeter of the sleeve for ease of access. The pockets may be reinforced to ensure that the clinician's fingers are securely received therein. The sleeve allows the clinician to move the baby's head while the sleeve is inflated and deployed.

Since the sleeve is to be used in medical applications, a sleeve material is selected that is suitable use for such an environment. The sleeve material must be sufficiently flexible to accommodate movement of the clinician's fingers during the procedure.

Described herein is a birth assist system that deploys a birth assist device having an inflatable sleeve, a pressure device fluidically coupled to the inflatable sleeve; a handle affixed to a proximal end of the inflatable sleeve; and a plurality of pockets configured to receive fingers of an operator at an outer perimeter of a distal end of the inflatable sleeve.

In one aspect, the pressure device is a pressure limited pump, wherein the pressure limited pump is affixed to the handle. In a further aspect, the birth assist system also has a switch to deflate the inflated sleeve. The birth assist system may optionally have a decompression valve, wherein the decompression valve has a predetermined set point pressure which is a predetermined maximum pressure for the inflated sleeve in operation. In one aspect, the decompression valve is a check valve.

Also described herein is a method for operating a birth assist device in which a birth assist device having at least an inflatable chamber disposed in an open-ended sleeve, a pressure device fluidically coupled to the inflatable chamber, and a handle affixed to a proximal end of the open-ended sleeve. In one aspect, the sleeve has an extender and one or more finger tabs disposed on its distal end. In another aspect, the sleeve has a plurality of pockets configured to receive a plurality of fingers of a clinician at an outer perimeter of a distal end of the sleeve. According to the described method, a positive pressure is introduced into the inflatable chamber with the pressure device. The clinician inserts at least one finger from one hand into one of the tabs or plurality of pockets of the birth assist device. In some aspects, the extender receives some portion of the clinician's other hand. These “holds” assist the clinician in guiding the sleeve over a portion of a head of a baby accessed through an incision in a mother's womb, thereby guiding the baby from the womb through the incision with the head of the baby secured by the inflatable chamber disposed in the sleeve.

In one aspect of the method, the pressure device is a pressure limited pump that may be compressed to either drive air into the inflatable sleeve or withdraw air from the inflatable sleeve.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise specifically noted, articles depicted in the drawings are not necessarily drawn to scale.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

1. A birth assist system comprising:
a birth assist device comprising:
an inflatable sleeve;
a pressure device fluidically coupled to the inflatable sleeve;
a handle affixed to a proximal end of the inflatable sleeve; and
a hand guide configured to receive fingers of an operator at an outer perimeter of a distal end of the inflatable sleeve.

2. The birth device of claim 1, wherein the hand guide is an extender configured to receive at least one finger of the operator, the extender comprising a base strip with ends affixed to the sleeve, at least one finger portion extending from the base strip and a gap between the sleeve and the extender that will receive a portion of an operator's hand.

3. The birth device of claim 2, wherein the extender comprises at least two finger portions.

4. The birth device of claim 3, wherein the extender comprises at least three finger portions.

5. The birth device of claim 2, wherein the extender is formed of silicone.

6. The birth device of claim 2, wherein the extender further comprises one or more bone strips disposed on at least one of the base strip a finger portion or both.

7. The birth device of claim 6, wherein the finger portion has at least one bone strip thereon.

8. The birth device of claim 7, wherein the finger portion has at least two bone strips thereon.

9. The birth device of claim 8, wherein the finger portion has a least three bone strips thereon.

10. The birth assist system of claim 1, wherein the pressure device is a pressure limited pump.

11. The birth assist system of claim 10, wherein the pressure limited pump is affixed to the handle.

12. The birth assist system of claim 11, further comprising a switch to deflate the inflated sleeve.

13. The birth assist system of claim 1, further comprising a decompression valve, wherein the decompression valve has a predetermined set point pressure which is a predetermined maximum pressure for the inflated sleeve in operation.

14. The birth assist system of claim 13, wherein the decompression valve is a check valve.

15. A method for operating a birth assist device in a Caesarean Section comprising;
providing a birth assist device comprising:
a birth assist device comprising:
an open-ended sleeve;
an inflatable chamber in a distal end of the open-ended sleeve;
a pressure device fluidically coupled to the inflatable chamber;
a handle affixed to a proximal end of the open-ended sleeve; and
a hand guide configured to receive a portion of a hand of an operator at an outer perimeter of a distal end of the open-ended sleeve;
providing positive pressure in the inflatable chamber with the pressure device;
inserting at least one finger from one hand into the hand guide;
guiding the open-ended sleeve over a portion of a head of a fetus accessed through an incision in a mother's womb;
inflating the inflatable chamber; and
guiding the fetus from the womb through the incision with the head of the fetus secured by the inflatable chamber.

16. The method of claim 15, wherein the hand guide is an extender configured to receive at least one finger of the operator, the extender comprising a base strip with ends affixed to the sleeve, at least one finger portion extending from the base strip and a gap between the sleeve and the extender that will receive a portion of an operator's hand.

17. The method of claim 16, wherein the extender comprises at least two finger portions.

18. The method of claim 17, wherein the extender comprises at least three finger portions.

19. The method of claim 16, wherein the extender is formed of silicone.

20. The method of claim 16, wherein the extender further comprises one or more bone strips disposed on at least one of the base strip a finger portion or both.

21. The method of claim 20, wherein the finger portion has at least one bone strip thereon.

22. The method of claim 21, wherein the finger portion has at least two bone strips thereon.

23. The method of claim 22 wherein the finger portion has a least three bone strips thereon.

24. The method of claim 15, wherein the pressure device is a pressure limited pump.

25. The method of claim 24, where the pressure limited pump is compressed to drive air into the inflatable chamber.

26. The method of claim 25, wherein the pressure limited pump comprises a switch, wherein the switch is actuated to withdraw air from the inflatable chamber.

27. The method of claim 16, further comprising, inserting a single finger into the extender.

28. The method of claim 27, further comprising guiding the inflatable chamber over a portion of the head of the fetus with individual fingers are inserted into individual pockets.

29. The method of claim 15, further comprising, inserting one or more fingers of one hand into one or more tabs attached to an exterior surface of the sleeve, wherein the one or more tabs are used to place the open-ended sleeve on an exposed portion of the head of the fetus, after which one or more fingers of the opposite hand are inserted into the hand guide; and after which the hand in the hand guide is used to move the sleeve further over the head of the fetus.

30. The method of claim 29, further comprising:
as the hand guide is used to guide the sleeve further over the head of the fetus, the hand guide moves off of the hand that was inserted into the hand guide and the hand, as it is removed from the hand guide, continues to advance the sleeve over the head of the fetus until the sleeve is in a position to inflate the inflatable chamber.
